(19) United States
(12) Patent Application Publication
(10) Pub. No.: US 2008/0216833 A1
(43) Pub. Date: Sep. 11, 2008

(54) Flow Sensing for Gas Delivery to a Patient

(76) Inventors: J. Raymond Pujol, Murrysville, PA (US); Andrew Alexander, (US); Christopher S. Lucci, Murrysville, PA (US)

Correspondence Address: MICHAEL W. HAAS;RESPIRONICS, INC., 1010 MURRY RIDGE LANE, MURRYSVILLE, PA, 15668, US

(21) Appl. No.: 12/040,043

(22) Filed: Feb. 29, 2008

Related U.S. Application Data
(60) Provisional application No. 60/905,340, filed on Mar. 7, 2007.

(51) Int. Cl. A61M 16/00

(52) U.S. Cl. 128/204.21

(57) ABSTRACT

A pressure support system that delivers a flow of gas to an airway of a patient. The system comprises a pressure generator, a torque monitor, a rotation monitor, and a processor. The pressure generator comprises an impeller and a motor. The impeller is coupled to the motor such that at least a portion of torque generated by the motor is provided to the impeller. As the impeller rotates through the body of breathable gas, the gas applies a torque to the impeller, and the impeller applies a force to the gas generating the gas flow. The torque monitor determines information related to the torque generated by the motor. The rotation monitor determines information related to a rotational velocity of the impeller and/or the motor. The processor determines one or more parameters of the flow of gas based on the information determined by the torque and rotation monitors.

### CROSS-REFERENCE TO RELATED APPLICATIONS

This application claims priority under 35 U.S.C. § 119(e) from provisional U.S. patent application No. 60/905,340 filed Mar. 7, 2007 the contents of which are incorporated herein by reference.

### BACKGROUND OF THE INVENTION

1. Field of the Invention

The invention relates to determinations of one or more parameters of a pressurized flow of breathable gas delivered by a pressure support system to a patient.

2. Description of the Related Art

It is well known to treat a breathing disorder, such as obstructive sleep apnea (OSA), with a pressure support device, such as a continuous positive airway pressure (CPAP) device. A CPAP device delivers a flow of fluid to the airway of the patient throughout the patient's breathing cycle in order to “splint” the airway, thereby preventing its collapse during sleep. The term “fluid” as used herein refers to any gas, including a gas mixture or a gas with particles, such as an aerosol medication, suspended therein. Most commonly, the fluid delivered to a patient by a pressure support system is pressured air. An example of such a CPAP device is the REMstar® family of CPAP devices manufactured by Respironics, Inc. of Murrysville, Pa.

Conventional pressure support devices generally rely on a sensor capable of directly measuring the flow rate, pressure, and/or volume of the flow of gas provided to a patient. However, such sensors add cost to these designs, and these sensors may be subject to deleterious operating conditions (e.g., moisture, heat, vibration, etc.). Other drawbacks with conventional techniques for determining parameters of a flow of gas being delivered to a patient by conventional pressure support devices also exist.

### SUMMARY OF THE INVENTION

One aspect of the invention relates to a pressure support system that delivers a pressurized flow of breathable gas to an airway of a patient. In one embodiment, the system comprises a pressure generator, a torque monitor, a rotation monitor, and a processor. The pressure generator comprises an impeller, also referred to as an impeller, and a motor. The motor is configured to generate a torque. The impeller is coupled to the motor such that at least a portion of the torque generated by the motor is provided to a rotor in the motor, which drives the impeller to rotate through a body of breathable gas. As the impeller rotates through the body of breathable gas the gas applies a torque to the impeller and the impeller applies a corresponding force to the body of breathable gas that generates a pressurized flow of breathable gas for delivery to a patient. The torque monitor is configured to determine information related to the torque generated by the motor. The rotation monitor is configured to determine information related to a rotational velocity of the impeller and/or the motor. The processor is configured to determine one or more parameters of the pressurized flow of breathable gas generated by the pressure generator based on the information determined by the torque monitor and the information determined by the rotation monitor. The determination of the one or more parameters of the pressurized flow of breathable gas includes an adjustment for at least a portion of a difference between the torque generated by the motor and the torque that is applied to the impeller by the body of breathable gas.

Another aspect of the invention relates to a method of delivering a pressurized flow of breathable gas to an airway of a patient. In one embodiment, the method comprises driving an impeller with a motor, the motor being configured to generate a torque, the impeller being coupled to the motor such that at least a portion of the torque generated by the motor is provided to a rotor in the motor, which drives the impeller to rotate through a body of breathable gas. As the impeller rotates through the body of breathable gas, the gas applies a torque to the impeller and the impeller applies a corresponding force to the gas that generates a pressurized flow of breathable gas for delivery to a patient. The method further includes determining information related to the torque that is generated by the motor; determining information related to a rotational velocity of the impeller and/or the motor; and determining one or more parameters of the pressurized flow of breathable gas generated by the rotation of the impeller based on the information related to the torque generated by the motor and information related to the rotational velocity of the impeller and/or the motor. The determination of the one or more parameters of the pressurized flow of breathable gas is made such that it includes an adjustment for at least a portion of a difference between the torque generated by the motor and the torque that is applied to the impeller by the body of breathable gas.

Another aspect of the invention relates to a pressure support system that delivers a pressurized flow of breathable gas to an airway of a patient. In one embodiment, the system comprises a pressure generator, a torque monitor, a rotation monitor, and a processor. The pressure generator comprises an impeller and a motor. The motor is configured to generate a torque. The impeller is coupled to the motor such that at least a portion of the torque generated by the motor is provided to a rotor in the motor, which drives the impeller through a body of gas. As the impeller rotates through the body of breathable gas, the gas applies a torque to the impeller and the impeller applies a corresponding force to the body of breathable gas that generates a pressurized flow of breathable gas for delivery to a patient. The torque monitor is configured to determine information related to the torque generated by the motor. The rotation monitor is configured to determine information related to a rotational velocity of the impeller and/or the motor.

The processor comprises a torque adjustment module, and a flow module. The torque adjustment module is configured to receive information related to the torque generated by the motor from the torque monitor, to determine information related to at least a portion of a difference between the torque generated by the motor and the torque that is applied to the impeller by the body of breathable gas, and to adjust information received from the torque monitor based on the information related to the at least a portion of a difference between the torque generated by the motor and the torque that is applied to the impeller by the body of breathable gas. The flow module is configured to determine a flow rate of the pressurized flow of breathable gas generated by the pressure generator based on the adjusted information related to the torque generated by the motor and the rotational velocity of the impeller and/or the motor.

Another aspect of the invention relates to a pressure support system that delivers a pressurized flow of breathable gas to an airway of a patient. In one embodiment of the invention, the system comprises means for driving an impeller with a motor, the motor being configured to generate a torque, the impeller being coupled to the motor such that at least a portion of the torque generated by the motor is provided to a rotor in the motor, which drives the impeller to rotate through a body of breathable gas. As the impeller rotates through the body of breathable gas the gas applies a torque to the impeller, and the impeller applies a corresponding force to the gas that generates a pressurized flow of breathable gas for delivery to a patient. The system also includes means for determining information related to the torque generated by the motor; means for determining information related to a rotational velocity of the impeller and/or the motor; and means for determining one or more parameters of the pressurized flow of breathable gas generated by the rotation of the impeller based on the information related to the torque generated by the motor and information related to the rotational velocity of the impeller and/or the motor. The determination of the one or more parameters of the pressurized flow of breathable gas is made such that it includes an adjustment for at least a portion of a difference between the torque generated by the motor and the torque that is applied to the impeller by the body of breathable gas.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an” and “the” include plural referents unless the context clearly dictates otherwise

### BRIEF DESCRIPTION OF THE DRAWINGS

FIG. 1 illustrates a pressure support system, in accordance with one or more embodiments of the invention;

FIG. 2 illustrates a flow, current, and speed curve, according to one or more embodiments of the invention; and

FIG. 3 is a flow chart illustrating a method of delivering a pressurized flow of breathable gas to a patient, in accordance with one or more embodiments of the invention.

### DETAILED DESCRIPTION OF THE EXEMPLARY EMBODIMENTS

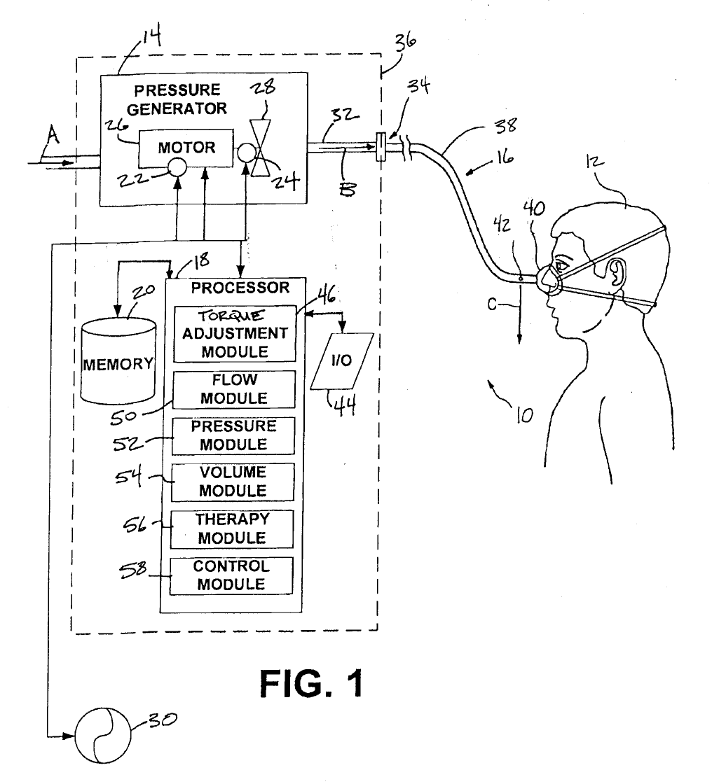

The general configuration of a pressure support system 10 in accordance with the principles of the present invention is described below with reference to FIG. 1. Pressure support system 10 is configured to provide a pressurized flow of breathable gas to a patient 12 according to a predetermined patient therapy algorithm. In order to deliver the pressurized flow of breathable gas to patient 12 according to a predetermined patient therapy algorithm, pressure support system 10 is capable of determining information related to the flow rate, pressure, and/or volume of the pressurized flow of breathable gas without the need for a sensor capable of directly measuring flow, pressure, and/or volume (e.g., as is required in conventional pressure support systems). However, it should be appreciated that in some embodiments, the determination of information related to one or more of these parameters without the need for a direct measurement of any of these parameters may be made in a system that also includes one or more sensors that directly measure other ones of these parameters.

In one embodiment, pressure support system 10 comprises a pressure generator 14, a patient circuit 16, a processor 18, and memory 20. Pressure generator 14 is operable to generate a pressurized flow of breathable gas that is delivered to patient 12 via patient circuit 16. Various aspects of the operation of pressure generator 14 may be monitored by one or more monitors (illustrated in FIG. 1 as a torque monitor 22 and a rotation monitor 24), as will be discussed later. Based on the aspects of the operation of pressure generator 14 monitored by the one or more monitors, processor 18 determines information related to the flow rate, pressure, and/or volume of the pressurized flow of breathable gas. Processor 18 utilizes the information related to the flow rate, pressure, and/or volume of the pressurized flow of breathable gas to control pressure generator 14 such that the pressurized flow of breathable gas is delivered to patient 12 according to a predetermined patient therapy algorithm.

Pressure generator 14 receives fluid, such as breathing gas, from a source of breathing gas, such as ambient atmosphere or a breathing gas storage tank or system, as indicated by arrow A, and elevates the pressure of the gas at its output. Pressure generator 14 includes a motor 26 and an impeller 28 coupled to the motor. In an exemplary embodiment, the impeller is coupled to the motor via a drive shaft. This coupling can be direct or indirect, e.g., through a gear assembly. The present invention also contemplates coupling the impeller directly to the motor, for example by forming the impeller with or fixing the impeller to the rotor in the motor. The combination of motor 26 and impeller 28 is often referred to as a blower.

During operation, motor 26 generates a torque. Impeller 28 is coupled to motor 26 such that at least a portion of the torque generated by motor 26 is provided to impeller 28. The torque thusly applied to impeller 28 drives the impeller to rotate through the body of breathable gas present within pressure generator 14. The present invention contemplates that impeller 28 can have a variety of configurations. One example of an impeller suitable for use in the present invention is disclosed in U.S. Pat. No. 6,622,724, the contents of which are incorporated herein by reference. The present invention further contemplates that impeller can be open or closed bladed, and can be configured to have a fan type of configuration and/or blade arrangement.

As impeller 28 rotates through the body of breathable bas, impeller 28 applies a corresponding force to the breathable gas (e.g., along arrow A), that compresses the breathable gas to generate the flow of pressurized breathable gas (e.g., indicated by arrow B). Thus, the motor applies torque on the impeller that is used to generate the pressurized flow of gas for delivery to the patient.

It should be further understood that the gas applies a torque to impeller 28 (e.g., along the blades of impeller 28). The torque applied to the impeller by the gas through which the impeller passes is related to items such as: (1) the fluid properties of the medium in which the impeller is immersed; (2) the flow and/or pressure conditions at the outlet and/or inlet of the blower, which are typically referred to as the “fluid load”; and (3) the blower design or configuration, such as the impeller geometry (blade shape, number, spacing, size, etc.), rotation direction of the impeller relative to the rest of the blower, and inlet and outlet geometries of the blower. Examples of fluid properties include temperature, humidity, pressure, gas composition, viscosity, mass/density, etc. (i.e., anything that affects the property of the medium through which the impeller passes). In addition, the fluid load is “seen” as a torque by the impeller and motor. The fluid load includes, for example, the patient induced pressure/flow changes at the outlet of the blower, changes in leak, or any other item that impacts pressure and flow at the outlet of the blower. All of these different torque components acting on the impeller/motor can be taken into consideration to determine one or more parameters associated with the pressurized flow of breathable gas delivered by the pressure support system to the patient, as discussed in detail below.

Motor 26 may include an A/C motor (e.g., single and/or poly phase induction including cage or wound rotor, synchronous machines including reluctance, wound field and permanent magnets, invariable reluctance machines including switch reluctance and stepper, etc.), or a D/C motor (e.g., a brushless motor, a coreless motor, etc.). Motor 26 may be driven by a current drawn from a power source 30 (e.g., an A/C or D/C source, such as a wall socket, a battery, etc.). By controlling the amount of current provided to motor 26 from power source 30, one or more aspects of the operation of motor 26 may be controlled. For example, the torque, the rotational velocity, the rotational acceleration, and/or other aspects of the operation of motor 26 may be controlled.

In one embodiment, patient circuit 16 includes a conduit 32 that carries fluid (e.g., the pressurized flow of breathable gas) from the output of pressure generator 14 to an external coupling, generally indicated at 34, on a housing 36 containing the components of pressure support system 10. Housing 36 is schematically illustrated in FIG. 1 by a dashed line surrounding the components of pressure support system 10 contained in housing 36. However, it should be appreciated that this illustration of housing 36 is not intended to be limiting. In other embodiments, more or fewer of the components of pressure support system 10 may be included within housing 36. In addition, multiple housings may be used to carry the indicated components. Patient circuit 16 also includes a conduit 38 that is attached to external coupling 34 of the housing 36. The patient circuit 16, in another embodiment, may alternately comprise a single conduit connected directly to the pressure generator 14 and extending to the pressure interface device 40 to be described below. Conduit 38 carries the pressurized flow of breathable gas from housing 36 to an airway of patient 12.

A patient interface device 40 at the end of patient circuit 16 communicates the pressurized flow of breathable gas in patient circuit 16 with the airway of patient 12. The present invention contemplates that patient interface device 40 is any device suitable for communicating an end of patient circuit 16 with the airway of patient 12. Examples of suitable patient interface devices include a nasal mask, oral mask or mouthpiece, nasal/oral mask, nasal cannula, trachea tube, intubation tube, hood or full face mask. It is to be understood that this list of suitable interface devices is not intended to be exclusive or exhaustive.

Because patient circuit 16 is illustrated as a single-limb circuit, i.e., it has only one conduit communicating the pressurized flow of breathable gas with the airway of patient 12, it includes an exhaust element 42 to exhaust gas, such as the patient's exhaled gas, from the otherwise closed system. This flow of exhaust gas is indicated by arrow C in FIG. 1. The present invention contemplates providing exhaust element 42 in patient circuit 16, on the patient interface device 40, or at both locations. Examples of conventional exhaust elements are described, for example, in U.S. Pat. No. Re. 35,339 to Rappoport; U.S. Pat. No. 5,937,855 to Serowski et al.; U.S. Pat. No. 6,112,745 to Lang; and published PCT application nos. WO 00/78381 to Gunaratnam et al. and WO 98/34665 to Kwok. These documents are incorporated by reference into this disclosure in their entirety.

It is to be understood that various other components may be provided in or coupled to patient circuit 16. For example, a bacteria filter, pressure control valve, flow control valve, sensor, meter, pressure filter, humidifier and/or heater can be provided in or attached to the patient circuit. Likewise, other components, such as sensors, muffler and filters can be provided at the inlet of pressure generator 14.

Although not shown in FIG. 1, the present invention also contemplates providing a secondary flow of gas in combination with the primary flow of breathable gas. For example, a flow of oxygen from any suitable source can be provided upstream to the inlet of pressure generator 14 or downstream of pressure generator 14 (e.g., in patient circuit 16 or at patient interface device 40) to control the fraction of inspired oxygen delivered to patient 12.

As was mentioned briefly above, one or more aspects of the operation of pressure generator 14 are monitored by one or more monitors. In the embodiment illustrated in FIG. 1, the one or more monitors include a torque monitor 22 and a rotation monitor 24. Torque monitor 22 monitors information related to the torque generated by motor 26. For example, in one embodiment, the information related to the torque generated by motor 26 includes information related to current drawn by motor 26 from power source 30 (e.g., current of one leg of motor 26, current of two legs of motor 26, current of three legs of motor 26, flux, etc.). Monitoring information related to the current drawn by motor 26 from power source 30 includes providing readings indicative of the amount of current being drawn at predetermined intervals (e.g., at a sampling rate). The amount of current drawn by motor 26 is related to the torque generated by motor 26.

Although various aspects of the invention are discussed below with respect to monitoring the current drawn by motor 26 as information related to the torque generated by motor 26, this is for illustrative purposes only. It is contemplated that other metrics related to the torque generated by motor 26 may be monitored. For example, a sensor capable of monitoring information related to force may be implemented to provide information related to the torque generated by motor 26. As another example, torque itself may be monitored either by an internal sensor provided within motor 26 or by a discrete sensor provided to generate readings of the torque generated by motor 26.

Rotation monitor 24 monitors information related to the rotation of motor 26 and/or impeller 28. Monitoring information related to the rotation of the motor 26 and/or impeller 28 may include providing readings of the rotational velocity of the motor 26 and/or impeller 28 at predetermined intervals. It should be appreciated that one or both of monitors 22 and 24 may or may not include separate hardware sensors incorporated into pressure generator 14. For example, in one embodiment, torque monitor 22 is an ammeter provided within system 10 to directly measure the current being supplied to motor 26, while in another embodiment torque monitor 22 is incorporated into a module within processor 18 that controls the supply of power to motor 26 (e.g., control module 58).

Processor 18 receives information related to the torque being generated by motor 26 and the rotation of motor 26 and/or impeller 28 from monitors 22 and 24, and based on this information, determines information related to the flow, pressure, and/or volume of the pressurized flow of breathable gas being delivered to patient 12. Processor 18 leverages the determined information to control pressure generator 14 to provide the pressurized flow of breathable gas according to one or more predetermined patient therapy algorithms.

As used herein, the term “predetermined patient therapy algorithm” refers to an algorithm for delivering the pressurized flow of breathable gas to patient 12 such that one or more of the flow, pressure, and/or volume of the pressurized flow of breathable gas are varied based on one or more factors. This may include varying one or more of the flow, pressure, and/or volume of the pressurized flow of breathable gas based on the respiration of patient 12 (e.g., triggering changes based on the inspiration and/or expiration of patient 12), based on predetermined timing intervals, based on a body position of patient 12, and/or based on one or more other predetermined occurrences or triggers. As another example, one or more of the flow, pressure, and/or volume of the pressurized flow of breathable gas may be varied in order to hold another one of the flow, pressure, and/or volume substantially constant (e.g., CPAP ventilation).

In another form, the respiratory treatment therapy, i.e., the patient therapy algorithm, involves providing a bi-level positive pressure therapy to the patient. In this treatment therapy, the pressure of fluid delivered to the patient's airway varies or is synchronized with the patient's breathing cycle to maximize the therapeutic effect and comfort to the patient. During inspiration, the patent receives an inspiratory positive airway pressure (IPAP), and during expiration, the patient receives an expiratory positive airway pressure (EPAP) that is lower than the IPAP. An example of a pressure support device that provides “bi-level” pressure support, in which a lower pressure is delivered to that patient during the patient's expiratory phase than during the inspiratory phase, is the BiPAP® family of devices manufactured and distributed by Respironics, Inc. of Pittsburgh, Pa. It should be noted that bi-level therapies can provide pressure waveforms having a variety of different patterns. For example, the pressure can be delivered in a traditional square wave or in a fashion that more closely mimics the pressure or flow waveform of a healthy human.

It is further known to provide a respiratory treatment therapy in which the pressure provided to the patient is automatically adjusted based on the detected conditions of the patient, such as whether the patient is snoring or experiencing an apnea, hypopnea, or snoring. This respiratory treatment technique is referred to as an auto-titration type of pressure support, because the pressure support device seeks to provide a pressure to the patient that is only as high as necessary to treat the disordered breathing. An example of a device that adjusts the pressure delivered to the patient based on whether or not the patient is snoring is the REMstar® Auto device manufactured and distributed by Respironics, Inc.

Other pressure support systems that offer other modes of providing positive pressure to the patient are also known. For example, a proportional assist ventilation (PAV®) mode of pressure support provides a positive pressure therapy in which the pressure of gas delivered to the patient varies with the patient's breathing effort to increase the comfort to the patient. U.S. Pat. Nos. 5,044,362 and 5,107,830 both to Younes, the contents of which are incorporated herein by reference, teach a pressure support device capable of operating in a PAV mode.

Proportional positive airway pressure (PPAP) devices deliver breathing gas to the patient based on the flow generated by the patient. U.S. Pat. Nos. 5,535,738; 5,794,615; 6,105,575; 6,609,517; and 6,932,084, (collectively referred to as “the PPAP patents”) the contents of which are incorporated herein by reference, teach a pressure support device capable of operating in a PPAP mode. Examples of devices that adjust the pressure delivered to the patient based on the patient's respiratory flow is the REMstar® Pro, Plus, or Auto with C-Flex™ or Bi-Flex® devices manufactured and distributed by Respironics, Inc. The term “C-Flex” refers to a device that provides a CPAP respiratory treatment therapy in which the pressure delivered to the patient is reduced in proportion to flow during expiration. The term “Bi-Flex” refers to a device that provides a bi-level respiratory treatment therapy in which either the IPAP or EPAP pressures are further reduced in proportion to flow.

It is also known to provide a combination of such respiratory therapies. For example, a CPAP device with C-Flex can be auto-titrating, such as REMstar® Auto with C-Flex™, so that the CPAP pressure varies during a treatment session based on the monitored condition of the patient. Similarly, a bi-level device with Bi-Flex can be auto-titrating, such as Bi-PAP® Auto with Bi-Flex™, so that the IPAP and EPAP pressures vary during a treatment session based on the monitored condition of the patient. In an auto titrating bi-level device, the difference between IPAP and EPAP, which is referred to as the pressure support (PS), can vary according to the auto-titration algorithm or it can be held constant depending on how the device is configured.

All of the above are examples of predetermined patient therapy algorithms and may include CPAP, bi-level ventilation, C-flex, A-flex, Bi-flex, auto-titration, proportional assist ventilation (PAV), or auto-servo ventilation or combinations thereof. In one embodiment, the predetermined patient therapy algorithm provides therapy designed to address one or more of apnea, hypopnea, flow limitation, Cheyne-Stokes respiration, and/or other respiratory phenomena. Operations included in the implementation of a predetermined patient therapy algorithm may include leak estimation, inhalation/exhalation state, triggering, patient circuit pressure, leak compensation algorithms, and/or other operations.

Memory 20 provides an electronically readable storage medium that is operatively coupled with processor 18. This operative couple is illustrated with an arrow in FIG. 1. Memory 20 may include optical readable storage media (e.g., optical disks, etc.), magnetically readable storage media (e.g., magnetic tape, magnetic hard drive, floppy drive, etc.), solid-state storage media (e.g., flash drive, etc.), and/or other electronically readable storage media. Memory 20 may store software algorithms, data, and/or other information that may enable processor 18 to function properly.

In one embodiment, system 10 includes a user interface 44 that provides an interface between processor 18 and an operator or patient 12. This enables information, data and/or instructions and any other communicable items, collectively referred to as “data”, to be communicated between a user and processor 18. This data may be communicated from user interface 44 to processor 18 by an operative communication link illustrated in FIG. 1 by an arrow. Examples of conventional input devices suitable for inclusion in interface 44 include a keypad, buttons, switches, or keyboard. Examples of conventional output devices suitable for inclusion in interface 44 include a display, lights or other visual indicia or an audio-based device, such as a speaker.

It is to be understood that other communication techniques, either hard-wired or wireless, are also contemplated by the present invention as interface 44. For example, the present invention contemplates providing a smart card terminal that enables data to be loaded into processor 18 from the smart card or loaded onto the smart card from the processor 18. Other exemplary interface devices and techniques adapted for use with the pressure support system 10 include, but are not limited to, an RS-232 port, CD reader/writer, DVD reader/writer, RF link, an IR link, modem (telephone, cable or other). In short, any technique for providing, receiving, or exchanging data with processor 18 are contemplated by the present invention as interface 44.

Returning to processor 18, it should be appreciated that although processor 18 is shown in FIG. 1 as a single entity, this is for illustrative purposes only. In some implementations, processor 18 may include a plurality of processing units. These processing units may be physically located within the same device (e.g., within housing 36 of system 10), or processor 18 may represent processing functionality of a plurality of devices operating in coordination. In instances in which a plurality of devices are implemented, operative communications links may be formed between the devices to enable communication and coordination therebetween. For example, in some embodiments, processor 18 may include one or more processors external to the other components of system 10 (e.g., a host computer), one or more processors that are included integrally in one or more of the components of system 10 (e.g., one or more processors included integrally within housing 36, etc.), or both. Processors external to other components within system 10 may, in some cases, provide redundant processing to the processors that are integrated with components in system 10, and/or the external processor(s) may provide additional processing to determine additional information related to the operation of system 10 and/or the pressurized flow of breathable gas delivered to patient 12.

As is shown in FIG. 1, in one embodiment processor 18 includes a torque adjustment module 46, a flow module 50, a pressure module 52, a volume module 54, a therapy module 56, and a control module 58. Modules 46, 50, 52, 54, 56, and/or 58 may be implemented in software, hardware, firmware, some combination of software, hardware, and/or firmware, and/or otherwise implemented. It should be appreciated that although modules 46, 50, 52, 54, 56, and/or 58 are illustrated in FIG. 1 as being co-located within a single processing unit, in implementations in which processor 18 includes multiple processing units modules 46, 50, 52, 54, 56, and/or 58 may be located remotely from the other modules and operative communication between modules 46, 50, 52, 54, 56, and/or 58 may be achieved via one or more communication links. Such communication links may be wireless or hard wired.

Torque adjustment module 46 adjusts information related to the torque generated by motor 26 that is provided by torque monitor 22. For example, in one embodiment, torque adjustment module 46 adjusts readings of current being drawn by motor 26 that are provided by torque monitor 22. As was mentioned above, the amount of current drawn by motor 26 is related to the torque generated by motor 26. This relationship may be expressed as:

T=K_{T}*I (1)

where T represents the torque generated by motor 26, K_{T} is related to the motor torque sensitivity constant, and I represents the current drawn by motor 26 from power source 30. The relationship expressed in equation (1) may be leveraged to determine a total amount of torque generated by motor 26 based on the total current drawn by motor 26. However, the parameters (e.g., flow rate, pressure, volume, etc.) of the pressurized flow of breathable gas may not be determined similarly (based on the total torque generated by motor 26) because not all of the torque generated by motor 26 is transferred to impeller 28 and/or to the pressurized flow of breathable gas. The adjustment of current readings and/or other metrics related to the torque generated by motor 26 made by torque adjustment module 46 enables enhanced determinations of one or more parameters of the pressurized flow of breathable gas from the information provided by torque monitor 22.

As motor 26 is driven to rotate impeller 28, the torque generated by motor 26 equals the sum of counteracting forces experienced by the motor 26/impeller 28 system. This may be roughly expressed as follows:

Tₘₒₜₒᵣ=T_{flow}+T_{windage}+T_{friction}+T_{acceleration}+Tₒₜₕₑᵣ, (2)

where Tₘₒₜₒᵣ represents the total torque generated by motor 26, T_{flow} represents the torque applied to impeller 28 by the flow of breathable gas as impeller 28 rotates through the gas (e.g., that corresponds to the compressive force that pressurizes the gas), T_{windage} represents the torque experienced by motor 26 due to windage created internally within motor 26, T_{friction} represents the torque dissipated due to friction/stiction in the motor 26/impeller 28 system, T_{acceleration} represents torque used to change the rotational velocity of impeller 28 (e.g., due to inertia), and Tₒₜₕₑᵣ represents the torque dissipated by one or more other torsions experienced by the motor 26/impeller 28 system. Tₒₜₕₑᵣ includes, for example, torques acting on impeller 28 such as: (1) the fluid properties of the medium in which the impeller is immersed; and (2) the blower design or configuration.

Due to the relationship between the torque created by motor 26 (Tₘₒₜₒᵣ) and the current drawn by motor 26 from power source 30 (Iₘₒₜₒᵣ) (illustrated by equation (1)), and based on equation (2), the current drawn by motor 26 from power source 30 (Iₜₒₜₐₗ) may be broken down into components that correspond to components of the total torque generated by motor 26 (Tₘₒₜₒᵣ). This is illustrated as follows:

Iₜₒₜₐₗ=I_{flow}+I_{windage}+I_{friction}+I_{acceleration}+Iₒₜₕₑᵣ, (3)

where Iₜₒₜₐₗ represents the total current drawn by motor 26, I_{flow} represents the current drawn by motor 26 to generate the portion of T_{flow}. I_{windage} represents the current drawn by motor 26 to generate a torque equal and opposite to T_{windage}, I_{friction} represents the current drawn by motor 26 to generate a torque equal and opposite to T_{friction}, I_{acceleration} represents the current drawn by motor 26 to generate a torque equal and opposite to T_{acceleration}, and Iₒₜₕₑᵣ represents the current drawn by motor 26 to generate a torque equal and opposite to Tₒₜₕₑᵣ.

In order to facilitate an enhanced determination of the flow rate, pressure, and/or volumetric measurement of the pressurized flow of breathable gas generated by the rotation of impeller 28 by motor 26 from the information provided by torque monitor 22 reflecting the total torque generated by motor 26 (e.g., Iₜₒₜₐₗ, in one embodiment), torque adjustment module 46 adjusts the information so that the adjusted information will more accurately correspond to the torque that is applied to the impeller by the breathable gas. For example, in an embodiment in which the torque monitor 22 provides information related to the current drawn by motor 26, the adjusted measurement of current will more closely reflect the component of the current that corresponds to the torque component associated with the compression of the pressurized flow of breathable gas by the rotation of impeller 28, or T_{flow} (this current component is represented by I_{flow} in equation (3)). Using the relationship between current and torque illustrated in equation (1), T_{flow} may be expressed in terms of I_{flow} as follows:

T_{flow}=K_{T}*I_{flow} (4)

In adjusting the current readings (of Iₜₒₜₐₗ) provided by torque monitor 22 to more closely coincide with I_{flow}, torque adjustment module 46 determines one or more of the current components drawn by motor 26 that correspond to at least a portion of the difference between the total amount of torque generated by motor 26, Tₘₒₜₒᵣ, and the torque applied by the breathable gas to impeller 28, T_{flow}. For example, these current components may include I_{windage}, I_{friction}, I_{acceleration}, etc. Torque adjustment module 46 then adjusts the readings of Iₜₒₜₐₗ provided by torque monitor 22 by removing the superfluous current components that have been determined (e.g., by subtracting I_{windage}, I_{friction}, and/or I_{acceleration} from Iₜₒₜₐₗ).

It should also be appreciated that due to the rotational nature of the motion of the motor 26/impeller 28, the torque generated by motor 26 used to change the rotational velocity of impeller 28 (T_{acceleration}) can be expressed as:

T_{acceleration}=Jₘ*α, (5)

where Jₘ is the moment of inertia of the motor 26/impeller 28 system; and α represents the angular acceleration of the motor 26/impeller 28 system during an increase or decrease in the rotational velocity of impeller 28. Similarly, leveraging the relationship illustrated in equation (1), T_{acceleration} may also be written as:

T_{acceleration}=K_{T}*I_{acceleration}. (6)

Combining equations 5 and 6 enables I_{acceleration} to be rewritten in terms of the angular acceleration of the motor 26/impeller 28 system (α) as follows:

I_{acceleration}=Jₘ/K_{T}*α. (7)

Torque adjustment module 46 leverages this relationship between α and I_{acceleration} to determine I_{acceleration} from a measurement or estimation of α. For example, in one embodiment, K_{T} and Jₘ may be known (e.g., via calibration, programmed at manufacture, etc.) and torque adjustment module 46 may determine I_{acceleration} according to equation (7) based on a measured or estimated α. In another embodiment, I_{acceleration} as a function of α may be determined empirically for a plurality of different α, and the results may be stored in a lookup table that is accessed by torque adjustment module 46 to determine I_{acceleration} based on a measured or estimated α.

In one embodiment, α is not directly measured. Instead, as in the embodiment illustrated in FIG. 1, system 10 includes only a monitor of the rotational velocity of impeller 28, and not its rotational acceleration. This is not intended to be limiting, as some embodiments include a rotational acceleration monitor that monitors the rotational acceleration of impeller 28 directly and provide readings of the rotational acceleration of impeller 28 to torque adjustment module 46 (e.g., at predetermined intervals). However, in embodiments in which the rotational acceleration of impeller 28 is not measured directly, an approximation or estimation of the instantaneous α of impeller 28 may be determined by torque adjustment module 46 based on readings of the rotational velocity of impeller 28 provided by rotation monitor 24. For example, the difference between two of the readings of rotational velocity (e.g., the two most recent readings) provided by rotation monitor 24 may be used as a measurement of α (e.g., ωₜ₋₁ωₜ∝α, where ωₜ represents rotational velocity at time t and ωₜ₋₁ represents the rotational velocity at time t−1). In some instances, this difference may be used in conjunction with the time interval between the two readings (e.g., the sampling period (T) to provide such a measurement (e.g.,

$$\frac{{ω}_{t - 1} - {ω}_{t}}{T} = α$$

In one embodiment, two or more measurements of α that have been made based on temporally proximate pairs of readings of rotational velocity may be averaged to determine a value representative of a of impeller 28. In one embodiment, determining a closed loop estimate of α may include comparing the rotational velocity of the motor 26/impeller 28 system to the time integral of an estimation of α (e.g., made according to one of the methods described above) to further refine the estimation of α.

In one embodiment, it is assumed that T_{windage} and T_{friction} (and the corresponding current components I_{windage} and I_{friction}) are negligible, and torque adjustment module 46 determines I_{acceleration} (e.g., as described above), or a corresponding component of some other metric of torque, and adjusts Iₜₒₜₐₗ, or a corresponding component of some other metric of torque, accordingly. However, it should be appreciated that this embodiment is not intended to be limiting, and that in other embodiments, one or both of I_{windage} and I_{friction}, or corresponding components of some other metric of torque, may similarly be determined and adjusted for. In order to adjust for I_{acceleration}, for example, torque adjustment module 46 may subtract the value determined for I_{acceleration} by torque adjustment module 46 from amount of the total current, Iₜₒₜₐₗ, indicated in a corresponding reading provided by torque monitor 22. This adjustment enables torque adjustment module 46 to provide an adjusted current that is approximately equal to the component of the current drawn by motor 26 that corresponds to the compressive force provided by impeller 28 to the pressurized flow of breathable gas, or I_{flow}.

It should be appreciated from equation (5) that as a of impeller 28 decreases, T_{acceleration} and the corresponding adjustment of Tₘₒₜₒᵣ made by torque adjustment module 46 will also become smaller. Therefore, during periods in which motor 26 is driving impeller 28 at a substantially constant speed, the impact of torque adjustment module 46 on determinations of the flow rate, pressure, and/or volume of the pressurized flow of breathable gas will become relatively small. However, when the speed of impeller 28 is changing, the adjustments made by torque adjustment module 46 will become larger and will significantly enhance the accuracy of determinations of the flow rate, pressure, and/or volume of the pressurized flow of breathable gas by system 10.

Flow module 50 is configured to determine a flow rate of the pressurized flow of breathable gas generated by the rotation of impeller 28. Flow module 50 determines the flow rate based on the principle that the flow rate of the flow of breathable gas is a function of the rotational velocity of impeller 28 and the compressive force that is applied to the gas by impeller 28 (e.g., the force applied to the gas by impeller 28 that corresponds to T_{flow}). In one embodiment, flow module 50 leverages the relationship between T_{flow} and I_{flow} (illustrated above in equation (1)) and the measurements of rotational velocity provided by rotation monitor 24 to determine the flow rate of the pressurized flow of breathable gas based on the measurements of rotational velocity and adjusted measurements of current drawn by motor 26 from power source 30 (e.g., determinations of I_{flow}) provided by current adjustment module 46.

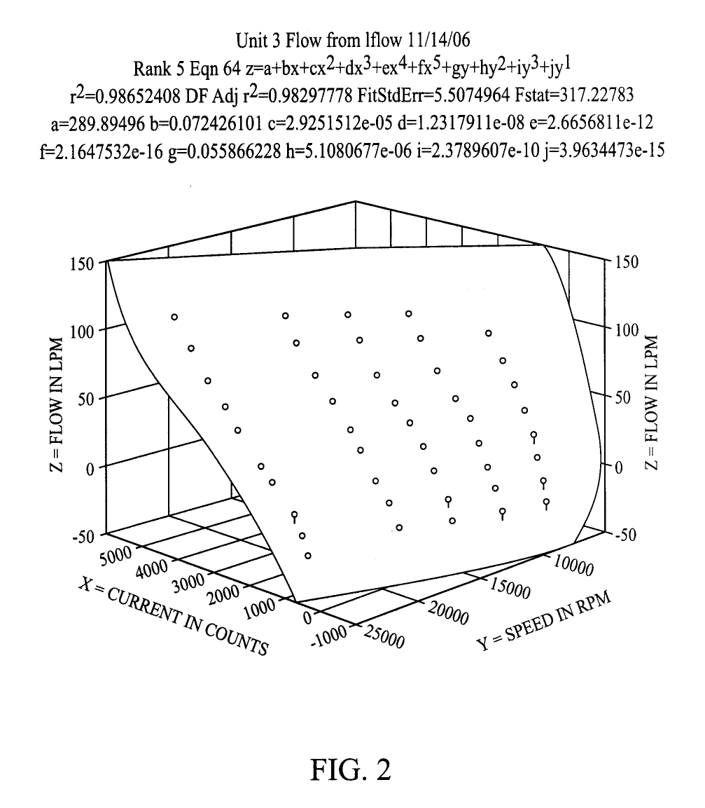

For example, flow module 50 includes a representation of an empirically predetermined current, velocity, and flow curve that returns a flow rate of the pressurized flow of breathable gas as a function of a measurement of rotational velocity of impeller 28 and an adjusted measurement of current (e.g., a determination of I_{flow}). The representation of the current, velocity, and flow curve may include a lookup table including values from the predetermined curve, a mathematical model of the predetermined curve, and/or other representations of a three variable function or curve. By way of example, FIG. 2 illustrates a current, velocity, and flow curve.

Returning to FIG. 1, pressure module 52 is configured to determine a pressure of the pressurized flow of breathable gas generated by the rotation of impeller 28. In one embodiment, pressure module 52 determines the pressure of the flow of breathable gas based on the flow rate of the gas (e.g., as determined by flow module 50). In determining the pressure of the flow of breathable gas based on the flow rate of the gas, pressure module 52 takes into account various variables of patient circuit 16. The variables of patient circuit 16 that are accounted for by pressure module 52 may include one or more of a circuit cross-section, a circuit path length, a geometry of an opening in patient circuit 16 (e.g., a size, a shape, etc.), a position on patient circuit 16 of an opening, one or more aspects of patient interface device 40, and/or other variables of patient circuit 16. It should be appreciated that first determining the flow rate of the pressurized flow of breathable gas, and then determining the pressure of the flow of breathable gas based on this flow rate, as described herein with respect to system 10, is not intended to be limiting. In some embodiments, pressure module 52 may determine the pressure of the flow of breathable gas directly from measurements of the rotational velocity of impeller 28 and adjusted measurements of the torque generated by motor 26 (or a related metric) using a predetermined curve (e.g., as described above with respect to flow module 50 using a predetermined flow, velocity, and current curve).

Volume module 54 is configured to determine a volumetric measurement of the pressurized flow of breathable gas generated by the rotation of impeller 28. In one embodiment, volume module 54 determines a volumetric measurement of the flow of breathable gas based on the flow rate of the gas (e.g., as determined by flow module 50). It should be appreciated, however, that first determining the flow rate of the pressurized flow of breathable gas, and then determining a volumetric measurement of the flow of breathable gas based on this flow rate, as described herein with respect to system 10, is not intended to be limiting. In some embodiments, volume module 54 may determine a volumetric measurement of the flow of breathable gas directly from measurements of the rotational velocity of impeller 28 and adjusted measurements of the torque generated by motor 26 (or a corresponding metric) using a predetermined curve (e.g., as described above with respect to flow module 50 using a predetermined flow, velocity, and current curve).

Therapy module 56 analyzes the parameters of the pressurized flow of breathable gas determined by one or more of flow module 50, pressure module 52, and/or volume module 54 to ensure that the flow of breathable gas is provided to patient 12 according to a predetermined patient therapy algorithm. In order to perform this functionality, therapy module 56 may analyze the relevant parameter(s) to determine information related to the respiration of patient 12 (e.g., the beginning and/or end of inspiration and/or expiration by patient 12, a respiratory event, the timing of respiration, other information related to the respiration of patient 12, etc.) and/or compare current levels of one or more of the measured parameters to desired levels of the one or more measured parameters. Based on this and other analysis, therapy module 56 determines adjustments that should be made in order to ensure that the flow of breathable gas is delivered in the appropriate manner. For example, therapy module 58 may determine that the flow rate and/or pressure of the flow of gas should be increased or decreased dynamically based on the breathing patterns of patient 12, the body position of patient 12, and/or other phenomena.

As was mentioned above, the predetermined patient therapy algorithm may include CPAP, bi-level, C-flex, A-flex, bi-flex, auto-titration, proportional assist ventilation positive airway pressure therapy, or auto-servo ventilation. In one embodiment, the predetermined patient therapy algorithm provides therapy designed to address one or more of apnea, hypopnea, flow limitation, Cheyne-Stokes respiration, and/or other respiratory phenomena. Operations included in the implementation of a predetermined patient therapy algorithm by therapy module 56 may include leak estimation, inhalation/exhalation state, triggering, patient circuit pressure, leak compensation algorithms, and/or other operations.

Control module 58 controls pressure generator 14 to provide the pressurized flow of breathable gas to patient 12 according to a predetermined patient therapy algorithm. In one embodiment, this includes controlling pressure generator 14 to provide adjustments to the flow rate, pressure, and/or volume of the pressurized flow of breathable gas that are relayed to control module 58 from therapy module 56 (e.g., determined as described above). The control module 58 controls the pressure generator 14 to provide the appropriate increases or decreases in the flow rate, pressure, and/or volume of the flow of breathable gas by, for example, controlling the amount of torque generated by motor 26 to increase or decrease the rotational velocity of impeller 28.

These increases and/or decreases in the rotational velocity of impeller 28 create periods during which the adjustment made by torque adjustment module 46 become non-trivial (for the reasons discussed above) in determining the one or more parameters of the flow of breathable gas. In pressure support systems that do not include the functionality of torque adjustment module 46, during these periods the inertia experienced by one or more components of a pressure generator may preclude an accurate determination of the one or more parameters of the flow of gas by these systems until the rotational velocity of impeller 28 “settles” to a relatively constant speed. This may cause a lag in these systems that impedes the implementation of the predetermined patient therapy algorithm.

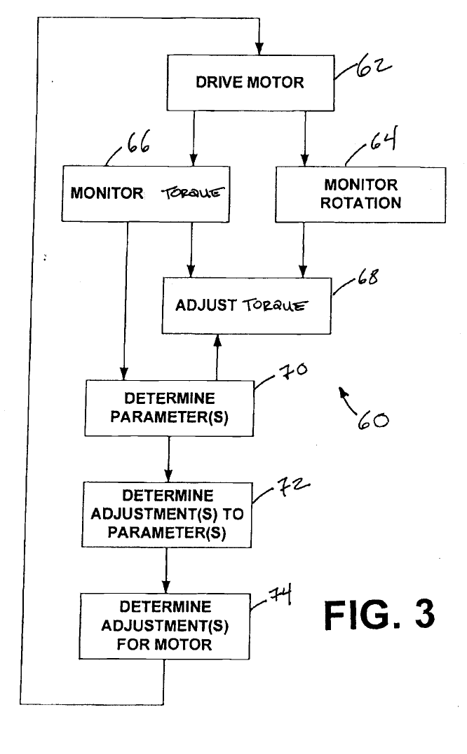

FIG. 3 includes a flow chart that illustrates a method 60 of delivering a pressurized flow of breathable gas to an airway of a patient, in accordance with one embodiment of the invention. It should be appreciated that although specific reference is made below regarding various operations of method 60 that can be executed by components of system 10 (e.g., illustrated in FIG. 1 and described above), this is for illustrative purposes only. In other embodiments, systems other than system 10 may be implemented to execute some or all of the operations of method 60.

Method 60 includes an operation 62, at which a motor generates a torque that drives the impeller to rotate through a body of breathable gas. As the impeller rotates through the body of breathable gas, the gas applies a torque to the impeller, and the impeller applies a corresponding compressive force to the body of breathable gas that generates a pressurized flow of breathable gas for delivery to the patient. The motor is driven by a current drawn from a power supply. In one embodiment, the motor may include a motor similar to motor 26 (e.g., illustrated in FIG. 1 and described above) and the impeller may include an impeller similar to impeller 28 (e.g., illustrated in FIG. 1 and described above).

At an operation 64, information related to the rotational velocity of the motor/impeller system may be determined. This may include providing periodic readings of the rotational velocity of the motor/impeller system. In one embodiment, operation 64 may be executed by a rotation monitor similar to rotation monitor 24 (e.g., illustrated in FIG. 1 and described above).

At an operation 66, information related to the torque being generated by the motor may be determined. This may include providing periodic readings of one or more metrics indicative of the amount of torque being generated by the motor (e.g., current, flux, force, torque, etc.). It should be noted that measuring flux includes measuring winding flux and/or magnet flux within the motor or external to the motor. If current is the monitored metric, it is to be understood that the current can be monitored in any one of a variety of ways, such as galvanic or flux based current measurement techniques. In one embodiment, operation 66 may be performed by a torque monitor similar to torque monitor 22 (e.g., illustrated in FIG. 1 and described above).

At an operation 68, a reading indicative of the torque being generated by the motor may be adjusted to account for at least a portion of a difference between the torque generated by the motor and the torque applied to the impeller by the breathable gas. This difference may include one or more of a portion of the differences caused by friction in the motor/impeller system, a portion of the difference caused by windage, a portion of the difference caused by acceleration and/or deceleration of the rotational velocity of the impeller, and/or other torques experienced by the motor/impeller system. In one embodiment, operation 68 includes (i) determining a component of the information related to the at least a portion of the difference between the torque generated by the motor and the torque applied to the impeller by the body of breathable gas and (ii) adjusting a reading of the information related to the total amount of torque generated by the motor by subtracting the determined component of the information related to at least a portion of the difference between the torque generated by the motor and the torque applied to the impeller by the body of breathable gas from the information related to the total amount of torque generated by the motor. In one embodiment, operation 68 is executed by a torque adjustment module similar to torque adjustment module 46 (e.g., shown in FIG. 1 and described above).

At an operation 70, one or more parameters of the pressurized flow of breathable gas are determined based on the information related to the adjusted torque determined at operation 68. Accordingly, the one or more parameters of the pressurized flow of breathable gas determined at operation 70 reflect an adjustment for at least a portion of the difference between the torque generated by the motor and the torque applied to the impeller by the body of breathable gas. In one embodiment, operation 70 includes the determination of one or more of a flow rate, a pressure, and/or a volume of the pressurized flow of breathable gas based on the adjusted information related to the torque generated by the motor and information related to the rotational velocity of the impeller (e.g., provided by operation 64). In one embodiment, operation 70 is executed by one or more of a flow module, a pressure module, and/or a volume module similar to flow module 50, pressure module 52, and/or volume module 54 (e.g., shown in FIG. 1 and described above).

Based on the one or more parameters of the pressurized flow of breathable gas determined at operation 70, the pressurized flow of breathable gas may be delivered according to a predetermined patient therapy algorithm. For example, at an operation 72, an adjustment of one or more parameters of the pressurized flow of breathable gas may be determined according to a predetermined patient therapy algorithm, and at an operation 74 an adjustment of one or more aspects of operation of the motor/impeller system (e.g., torque, rotational velocity, etc.) to provide the adjustment determined at operation 72 may be determined. The adjustment of the one or more aspects of operation of the motor/impeller system determined at operation 74 may then be implemented in operation 62. In one embodiment, operations 72 and 74 may be executed by a therapy module and a control module, respectively, similar to therapy module 56 and control module 58, respectively (e.g., shown in FIG. 1 and described above).

The present invention has been described above with respect to rotational motors. It is to be understood that this invention also applies to linear motors. In which case, torque would be expressed as a force, but all other aspects of the invention would be applicable to the use of a linear motor. Moreover, the term “impeller” is intended to encompass any structure that is suitable for moving fluid, whether rotary, linear, or other configuration.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A pressure support system that delivers a pressurized flow of breathable gas to an airway of a patient, the system comprising:
a pressure generator comprising an impeller and a motor, the motor being configured to generate a torque, the impeller being coupled to the motor such that at least a portion of the torque generated by the motor is provided to the impeller to drive the impeller to rotate through a body of breathable gas, wherein as the impeller rotates through the body of breathable gas the gas applies a torque to the impeller and the impeller applies a corresponding force to the body of breathable gas that generates a pressurized flow of breathable gas for delivery to a patient;
a torque monitor configured to determine information related to the torque generated by the motor;
a rotation monitor configured to determine information related to a rotational velocity of the impeller and/or the motor; and
a processor configured to determine one or more parameters of the pressurized flow of breathable gas generated by the pressure generator based on the information determined by the torque monitor and the information determined by the rotation monitor, wherein the determination of the one or more parameters of the pressurized flow of breathable gas includes an adjustment for at least a portion of a difference between the torque generated by the motor and the torque that is applied to the impeller by the body of breathable gas.

2. The pressure support system of claim 1, wherein the adjustment for at least a portion of the difference between the torque generated by the motor and the torque that is applied to the impeller by the body of breathable gas comprises one or more of at least a portion of the difference caused by windage, a portion of the difference caused by friction, or a portion of the difference caused by one or more changes in the rotational velocity of the impeller.

3. The pressure support system of claim 1, wherein the one or more parameters of the pressurized flow of breathable gas comprises one or more of a pressure, a flow rate, or a volume of the pressurized flow of breathable gas.

4. The pressure support system of claim 1, wherein the motor draws an amount of current to generate torque, and wherein the information related to the torque generated by the motor comprises information related to the amount of current drawn by the motor.

5. The pressure support system of claim 1, wherein the processor is configured to adjust the determination of the one or more parameters of the flow of breathable gas by:
(a) determining information related to at least a portion of the difference between the torque generated by the motor and the torque that is applied to the impeller by the body of breathable gas,
(b) adjusting the information related to the total amount of torque generated by the motor based on the determined information related to the at least a portion of the difference between the torque generated by the motor and the torque that is applied to the impeller by the body of breathable gas, and
(c) determining the one or more parameters of the flow of breathable gas generated by the pressure generator based on the adjusted information related to the torque generated by the motor and the information related to the rotational velocity of the impeller and/or the motor.

6. The pressure support system of claim 5, wherein the processor is configured to determine information related to the rate of change of the rotational velocity of the impeller and/or the motor, and wherein determinations by the processor of information related to the difference between the torque generated by the motor and the torque that is applied to the impeller by the body of breathable gas are made based on the determined information related to the rate of change of the rotational velocity of the impeller and/or the motor.

7. The pressure support system of claim 1, wherein the processor is configured to implement determinations of the one or more parameters of the pressurized flow of breathable gas to control the pressure generator to provide the pressurized flow of breathable gas to the patient according to a predetermined patient treatment algorithm.

8. The pressure support system of claim 7, wherein the predetermined patient treatment algorithm comprises one or more of CPAP, C-flex, bi-flex, A-flex, bi-level, auto-titration, auto-servo ventilation, or proportional assist ventilation positive airway pressure therapy.

9. The pressure support system of claim 7, wherein the predetermined patient treatment algorithm comprises therapy for one or more of apnea, hypopnea, flow limitation, or Cheyne-Stokes respiration.

10. The pressure support system of claim 7, wherein controlling the pressure generator to provide the pressurized flow of breathable gas to the patient according to a predetermined patient treatment comprises implementing one or more of leak estimation, inhalation/exhalation state, triggering, patient circuit pressure, or leak compensation algorithms.

11. A method of delivering a pressurized flow of breathable gas to an airway of a patient, the method comprising:
driving an impeller with a motor, the motor being configured to generate a torque, the impeller being coupled to the motor such that at least a portion of the torque generated by the motor is provided to the impeller to drive the impeller to rotate through a body of breathable gas, wherein as the impeller rotates through the body of breathable gas the gas applies a torque to the impeller and the impeller applies a corresponding force to the gas that generates a pressurized flow of breathable gas for delivery to a patient;
determining information related to the torque that is generated by the motor;
determining information related to a rotational velocity of the impeller and/or the motor; and
determining one or more parameters of the pressurized flow of breathable gas generated by the rotation of the impeller based on the information related to the torque generated by the motor and information related to the rotational velocity of the impeller and/or the motor, wherein the determination of the one or more parameters of the pressurized flow of breathable gas is made such that it includes an adjustment for at least a portion of a difference between the torque generated by the motor and the torque that is applied to the impeller by the body of breathable gas.

12. The method of claim 11, wherein the gas adjustment for at least a portion of the difference between the torque generated by the motor and the torque that is applied to the impeller by the body of breathable gas comprises one or more of at least a portion of difference caused by windage, at least a portion of the difference caused by friction, or at least a portion of the difference caused by one or more changes in the rotational velocity of the impeller and/or the motor.

13. The method of claim 11, wherein the one or more parameters of the pressurized flow of breathable gas comprise one or more of a flow rate, a pressure, or a volume of the pressurized flow of breathable gas.

14. The method of claim 11, wherein the motor draws an amount of current to generate torque, and wherein the information related to the torque generated by the motor comprises information related to the amount of current drawn by the motor.

15. The method of claim 11, wherein determining the one or more parameters of the pressurized flow of breathable gas such that it reflects an adjustment for an amount of force provided to the impeller by the motor that is not transferred from the impeller to the pressurized flow of breathable gas that pressurizes the gas comprises:
determining information related to at least a portion of the difference between the torque generated by the motor and the torque that is applied to the impeller by the body of breathable gas;
adjusting the information related to the total torque generated by the motor based on the determined information related to the at least a portion of the difference between the torque generated by the motor and the torque that is applied to the impeller by the body of breathable gas; and
determining the one or more parameters of the flow of breathable gas generated by the pressure generator based on the adjusted information related to the torque by the motor and the information related to the rotational velocity of the impeller and/or the motor.

16. The method of claim 15, further comprising determining information related to the rate of change of the rotational velocity of the impeller and/or the motor, wherein determining the information related to the at least a portion of the difference between the torque generated by the motor and the torque that is applied to the impeller by the body of breathable gas is based on the determined information related to the rate of change of the rotational velocity of the impeller and/or the motor.

17. The method of claim 11, further comprising controlling the motor to provide the pressurized flow of breathable gas to the patient according to a predetermined patient treatment algorithm.

18. The method of claim 17, wherein the predetermined patient treatment algorithm comprises one or more of CPAP, C-flex, bi-flex, a-flex, bi-level, auto-titration, proportional assist ventilation positive airway pressure, or auto-servo ventilation.

19. The method of claim 17, wherein the predetermined patient treatment algorithm comprises therapy for one or more of apnea, hypopnea, flow limitation, or Cheyne-Stokes respiration.

20. The method of claim 17, wherein controlling the pressure generator to provide the pressurized flow of breathable gas to the patient according to a predetermined patient treatment comprises implementing one or more of leak estimation, inhalation/exhalation state, triggering, patient circuit pressure, or leak compensation algorithms.

21. A pressure support system that delivers a pressurized flow of breathable gas to an airway of a patient, the system comprising:
a pressure generator comprising an impeller and a motor, the motor being configured to generate a torque, the impeller being coupled to the motor such that at least a portion of the torque generated by the motor is provided to the impeller to drive the impeller through a body of gas, wherein as the impeller rotates through the body of breathable gas the gas applies a torque to the impeller and the impeller applies a corresponding force to the body of breathable gas that generates a pressurized flow of breathable gas for delivery to a patient;
a torque monitor configured to determine information related to the torque generated by the motor;
a rotation monitor configured to determine information related to a rotational velocity of the impeller and/or the motor;
a processor comprising:
a torque adjustment module configured to receive information related to the torque generated by the motor from the torque monitor, to determine information related to at least a portion of a difference between the torque generated by the motor and the torque that is applied to the impeller by the body of breathable gas, and to adjust information received from the torque monitor based on the information related to the at least a portion of a difference between the torque generated by the motor and the torque that is applied to the impeller by the body of breathable gas; and
a flow module configured to determine a flow rate of the pressurized flow of breathable gas generated by the pressure generator based on the adjusted information related to the torque generated by the motor and the rotational velocity of the impeller and/or the motor.

22. The pressure support system of claim 21, wherein the adjustment for at least a portion of the difference between the torque generated by the motor and the torque that is applied to the impeller by the body of breathable gas comprises one or more of at least a portion the difference caused by windage, at least a portion of the difference caused by friction, or at least a portion of the difference caused by one or more changes in the rotational velocity of the impeller and/or the motor.

23. The pressure support system of claim 21, wherein the motor draws an amount of current to generate torque, and wherein the information related to the torque generated by the motor comprises information related to the amount of current drawn by the motor.

24. The pressure support system of claim 21, wherein the torque adjustment module determines the at least a portion of the difference between the torque generated by the motor and the torque that is applied to the impeller by the body of breathable gas based on a rate of change of the rotational velocity of the impeller and/or the motor.

25. The pressure support system of claim 21, wherein the processor further comprises a control module that is configured to control one or more aspects of the operation of the pressure generator such that the pressurized flow of breathable gas is delivered to the patient according to a predetermined patient treatment algorithm.

26. The pressure support system of claim 25, wherein the predetermined patient treatment algorithm comprises one or more of CPAP, C-flex, A-flex, bi-flex, bi-level, auto-titration, proportional assist ventilation positive airway pressure therapy, or auto-servo ventilation.

27. The pressure support system of claim 25, wherein the predetermined patient treatment algorithm comprises therapy for one or more of apnea, hypopnea, flow limitation, or Cheyne-Stokes respiration.

28. The pressure support system of claim 25, wherein controlling the pressure generator to provide the pressurized flow of breathable gas to the patient according to a predetermined patient treatment comprises implementing one or more of leak estimation, inhalation/exhalation state, triggering, patient circuit pressure, or leak compensation algorithms.

29. A pressure support system that delivers a pressurized flow of breathable gas to an airway of a patient, the system comprising:
means for driving an impeller with a motor, the motor being configured to generate a torque, the impeller being coupled to the motor such that at least a portion of the torque generated by the motor is provided to the impeller to drive the impeller to rotate through a body of breathable gas, wherein as the impeller rotates through the body of breathable gas the gas applies a torque to the impeller and the impeller applies a corresponding force to the gas that generates a pressurized flow of breathable gas for delivery to a patient;
means for determining information related to the torque generated by the motor;
means for determining information related to a rotational velocity of the impeller and/or the motor;
means for determining one or more parameters of the pressurized flow of breathable gas generated by the rotation of the impeller based on the information related to the torque generated by the motor and information related to the rotational velocity of the impeller and/or the motor, wherein the determination of the one or more parameters of the pressurized flow of breathable gas is made such that it includes an adjustment for at least a portion of a difference between the torque generated by the motor and the torque that is applied to the impeller by the body of breathable gas.

30. The system of claim 29, wherein the gas adjustment for at least a portion of the difference between the torque generated by the motor and the torque that is applied to the impeller by the body of breathable gas comprises one or more of at least a portion of difference caused by windage, at least a portion of the difference caused by friction, or at least a portion of the difference caused by one or more changes in the rotational velocity of the impeller and/or the motor.

31. The system of claim 29, wherein the one or more parameters of the pressurized flow of breathable gas comprise one or more of a flow rate, a pressure, or a volume of the pressurized flow of breathable gas.

32. The system of claim 29, wherein the motor draws an amount of current to generate torque, and wherein the information related to the torque generated by the motor comprises information related to the amount of current drawn by the motor.

33. The system of claim 29, wherein determining the one or more parameters of the pressurized flow of breathable gas such that it reflects an adjustment for an amount of force provided to the impeller by the motor that is not transferred from the impeller to the pressurized flow of breathable gas comprises:
determining information related to at least a portion of the difference between the torque generated by the motor and the torque that is applied to the impeller by the body of breathable gas;
adjusting the information related to the total torque generated by the motor based on the determined information related to the at least a portion of the difference between the torque generated by the motor and the torque that is applied to the impeller by the body of breathable gas; and
determining the one or more parameters of the flow of breathable gas generated by the pressure generator based on the adjusted information related to the torque generated by the motor and the information related to the rotational velocity of the impeller and/or the motor.

34. The system of claim 33, further comprising means for determining information related to the rate of change of the rotational velocity of the impeller and/or the motor, wherein determining the information related to the at least a portion of the difference between the torque generated by the motor and the torque that is applied to the impeller by the body of breathable gas is based on the determined information related to the rate of change of the rotational velocity of the impeller and/or the motor.

35. The system of claim 29, further comprising means for controlling the motor to provide the pressurized flow of breathable gas to the patient according to a predetermined patient treatment algorithm.

36. The system of claim 35, wherein the predetermined patient treatment algorithm comprises one or more of CPAP, C-flex, bi-flex, A-flex, bi-level, auto-titration, proportional assist ventilation positive airway pressure therapy, or auto-servo ventilation.

37. The system of claim 35, wherein the predetermined patient treatment algorithm comprises therapy for one or more of apnea, hypopnea, flow limitation, or Cheyne-Stokes respiration.

38. The system of claim 35, wherein controlling the pressure generator to provide the pressurized flow of breathable gas to the patient according to a predetermined patient treatment comprises implementing one or more of leak estimation, inhalation/exhalation state, triggering, patient circuit pressure, or leak compensation algorithms.
